(19) United States
(12) Patent Application Publication
(10) Pub. No.: US 2026/0108489 A1
(43) Pub. Date: Apr. 23, 2026

(54) TOPICAL NASAL PHARMACEUTICAL COMPOSITION FOR THE TREATMENT AND PROPHYLAXIS OF CHRONIC RHINOSINUSITIS (CRS)

(71) Applicants: Eduardo Roberto Varizat, Río Cuarto (AR); Daniel Roberto Varizat, Río Cuarto (AR)

(72) Inventors: Eduardo Roberto Varizat, Río Cuarto (AR); Daniel Roberto Varizat, Río Cuarto (AR)

(21) Appl. No.: 19/359,380

(22) Filed: Oct. 15, 2025

(30) Foreign Application Priority Data
Oct. 23, 2024 (AR) P20240102851

(51) Int. Cl. A61K 31/395; A61K 9/00; A61K 9/08; A61K 31/4174; A61K 31/56; A61K 39/02; A61K 39/09; A61K 39/102; A61K 39/108; A61P 31/04; A61P 37/04

(52) U.S. Cl. CPC A61K 31/395; A61K 9/0043; A61K 9/08; A61K 31/4174; A61K 31/56; A61K 39/0208; A61K 39/0266; A61K 39/092; A61K 39/102; A61P 31/04; A61P 37/04

(57) ABSTRACT

The invention relates to a new topical nasal pharmaceutical composition for the treatment and prophylaxis of Rhinosinusitis (RS), comprising a homogeneous aqueous suspension with a therapeutically effective amount of the following well-known active ingredients: Rifamycin, an antibiotic and anti-inflammatory agent for topical or local use; fluticasone propionate, an anti-inflammatory agent for local use; optionally, oxymetazoline hydrochloride, a decongestant; and bacterial antigens, local immunostimulants; 0.9% saline solution, a vehicle; and pharmaceutically acceptable excipients q.s., which are formulated for topical administration via nasal spray. The combination of these compounds produces synergy, resulting in a greater local antibiotic, anti-inflammatory, decongestant and immunomodulatory effect at the specific site of action. Its effectiveness in chronic rhinosinusitis is particularly notable in low doses. So is its good tolerance, absence of adverse effects, and ease of application. No rebound congestion has been reported with prolonged use.

### FIELD OF THE INVENTION

The present invention relates to the field of nasal decongestant agents, particularly to topical nasal pharmaceutical compositions. More particularly, the present invention relates to a topical nasal pharmaceutical composition for the treatment and prophylaxis of Chronic Rhinosinusitis (CRS).

### BACKGROUND OF THE INVENTION

It is well-known in an endless number of research and scientific works written by different international authors, that Chronic Rhinosinusitis (CRS) does not have an effective and definitive treatment for a significant amount of cases at the present time. There follow some examples of what we highlight, “A complex combination of inflammatory and microbial associations underlies chronic inflammatory condition Chronic Rhinosinusitis (CRS), and the etiology remains poorly understood.” (Michael Hoggard, Sharon Waldvogel-Thurlow, Melissa Zoing, Kevin Chang, Fiona J. Radcliff, Brett Wagner Mackenzie, Kristi Biswas, Richard G Douglas, Michael W Taylor. Inflammatory Endotypes and Microbial Associations in Chronic Rhinosinusitis. Front. Immunol., 18 Sep. 2018. Sec. Inflammation. Vol. 9-2018. doi.org/10.3389/fimmu.2018.02065).

“The pathogenesis of CRS is still unclear due to its high heterogeneity.” (Yuanqiong He, Yijie Fu, Yuqi Wu, Tianmin Zhu, Hui Li) Pathogenesis and treatment of chronic rhinosinusitis from the perspective of sinonasal epithelial dysfunction. Front. Med., 16 Apr. 2023. Sec. Pathology. Vol. 10-2023. doi.org/10.3389/fmed.2023.1139240).

“Chronic rhinosinusitis (CRS) is a prevalent inflammatory disease. No medication is approved by the U.S. Food and Drug Administration (FDA) for the most common form, CRS without nasal polyps (also called chronic sinusitis).” (James N. Palmer, Nithin D. Adappa, Rakesh K. Chandra, Greg E. Davis, Mahboobeh Mahdavinia, John Messina, Randall A. Ow, Zara M. Patel, Anju T. Peters, Harry Sacks, Rodney J. Schlosser, Raj Sindwani, Zachary M. Soler, Andrew A. White, Sarah K. Wise, and Ramy A. Mahmoud.) Efficacy of EDS-FLU for Chronic Rhinosinusitis: Two Randomized Controlled Trials (ReOpen1 and ReOpen2). J Allergy Clin Immunol Pract. April 2024. Vol. 12, No. 4, pages 1049-1061).

These definitions given by different authors prove that CRS is still an unsolved problem for researchers, and thus there is no defined effective treatment therefor. This causes patients to wander around the offices of general practitioners and pediatricians up to ENT or allergy specialists, failing to found any convincing solutions for this condition.

The probable reasons for these problems in obtaining good treatment results lie on the fact that the etiology of the condition responds to countless causes, often interrelated, which result in persistent or chronic inflammation cases. Among those mostly recognized, it is possible to recite damage to the epithelial barrier, which is caused by multiple factors, such as: different types of microbes, environmental pollution, aeroallergens, environmental thermal factors, different workspaces and jobs, genetic predisposition, and epigenetics among the most considered factors. When the epithelial barrier is damaged due to one or more of the aforementioned causes, it favors contact with and activation of various “innate immunity” components—phagocytes, leukocytes, antigen-presenting cells (APCs), interleukins, among others—the consequence of which is inflammation. This inflammatory situation facilitates the activation of various “adaptive immunity,” mechanisms which are frequently activated and converge in the same patient, resulting in further inflammation. Each mechanism acts with inflammatory processes that are inherent thereto, involving different cellular components and producing different cytokines. On the other hand, in this complex situation, the nervous system is also activated through its peripheral nerve endings with receptors that stimulate the release of neuropeptides, which in turn act on immune cells, activating them. All of this results in multi-causal and persistent inflammation.

With the aforementioned problems, infectious complications and hyperreactive reactions such as polyposis, though less frequent, are common. Based on the above, these multi-causal features of the etiopathogenesis turn “Chronic Rhinosinusitis” into a “Complex Chronic Disease,” with frequent relapses and treatments with variable and sometimes disappointing results, even those considered the most effective.

Combinations of antibiotic and/or anti-inflammatory agents and/or decongestants and/or immunomodulators containing more than one active ingredient are widely used in therapeutics for oral and/or nasal administration. Many of these combinations are formulated in an attempt to produce greater and better effects than the administration of their active ingredients alone. The response phenomenon is explained by the synergy achieved by the combination of the active ingredients, which enhances their activity. An example of this can be seen in allowed Patent MXPA/A/2001/005164, entitled: “Topical pharmaceutical composition comprising an antihistamine and a vasoconstrictor in nasal spray” (World-Trade Import-Export, Wtie, A.G) (CIP A61K 9/06, A61P 37/08, A61P 9/10).

Said patent refers to the formulation or combination of two compounds, one them being an antihistamine known as azelastine and the other a vasoconstrictor known as oxymetazoline, formulated as a nasal spray for topical application. The combination of these compounds produces synergy, resulting in a greater anti-allergy, anti-inflammatory, and decongestant effect at the specific site of action. Despite certain similarities with the composition of the present patent application, namely, some similar therapeutic effects, nasal mode of use and composition comprising anti-inflammatory and decongestant active ingredients, there are remarkable differences with said patent and the rest of the patents analyzed [WO2017095210—Combination of oxymetazoline and ipratropium in topical nasal application for the treatment of cough].

Contrary to the patents cited above, the pharmaceutical composition of the present patent application is different not only on the basis of the greater combination of active ingredients it comprises but also of the varying therapeutic effects thereof, the inclusion of Rifamycin being a novel and unprecedented factor, especially due to the local immunomodulatory effects detailed below. For these reasons, it is highly effective in treating chronic rhinosinusitis. Generally, chronic rhinosinusitis is understood as mucositis, which is an inflammation of the epithelium or mucosa of the nose and paranasal sinuses. The most common therapies for treating mucositis, including chronic rhinosinusitis, involve local and systemic steroids, anti-inflammatory agents, decongestants, antihistamines, systemic antibiotics, and in some cases, surgical treatment is attempted, along with other less common therapies.

However, such therapies do not address the local infectious components of mucositis.

Surprising evidence has been found in the administration via nasal spray of a composition that incorporates a topical antibiotic agent such as Rifamycin in association with other local decongestant, anti-inflammatory and immunomodulatory agents, which favor reduction or interruption of the process associated with mucositis that is typical of Chronic Rhinosinusitis.

Druce (1993, Immunol Allergy Clinics N. Am. 13:119-132) reviews agents, strategies and diagnostic techniques, treatments and problems related to sinusitis. A protocol for the treatment of chronic sinus-related symptoms included (1) amoxicillin or trimethoprim-sulfamethoxazole, (2) beclomethasone aqueous nasal spray, (3) guaifenesin-pseudoephedrine, (4) steam inhalation, and (5) saline nasal spray or irrigation.

Oliveira et al. (1997, Ann. Allergy Asthma Immunol. 79:70-74) describe a study demonstrating that children with allergic rhinitis and sinusitis suffering from asthma improve their non-specific bronchial hyperreactivity to methacholine exposure and reduce their sinusitis-related symptoms after 30 days with concurrent administration of intranasal saline, prednisone, sulfamethoxazole and trimethoprim, azatadine, and pseudoephedrine.

Given the constant patient consultations and the limitations and inadequacy of the standard therapeutic tools used to treat cases of chronic rhinosinusitis, a topical pharmaceutical composition aimed at the prophylaxis and control of microbes in the nasal cavities was tested with great effectiveness.

The premise is that microbes in the local microenvironment must play an important role in the pathology due to their constant contact with the rhinosinusal respiratory mucosa. In addition, given any compromise of epithelial integrity due to various reasons, they cause inflammation owing to infection. In other situations (as long as this compromise of epithelial integrity persists), they trigger a persistent and exaggerated defensive inflammatory reaction called “hyperreactivity” by the immune system. Therefore, it is interpreted that the inflammation of Chronic Rhinosinusitis, in a large number of cases, is a product or consequence of the presence of local bacteria common in the rhinosinusal microenvironment.

Accordingly, in the composition that is the object of the present application, combating bacterial presence turns out to be of utmost importance through the inclusion of a topical antibiotic such as Rifamycin in the pharmaceutical composition, normally used for other therapeutic purposes. This is how “Rifamycin,” an antibiotic for local use, is incorporated into the composition.

This is a novel and unprecedented improvement, since this antibiotic was not intended for topical use in the rhinosinusal cavity in the form of a spray. To this end, the scientific literature based on the use of rifamycin was reviewed, namely:

“Rifamycin has been used for oropharyngeal treatment of diphtheria carriers ( . . . ) General organ tolerance is very good ( . . . ) Intracavitary application in abscesses, suppurations, fistulas, or even intrapleural administration is characterized by good local and general tolerance. The use thereof has become widespread.” (Bergoglio, 1993:288; Antibióticos—5th Edition, Editorial Panamericana).

Considering that the combination with anti-inflammatory agents could enhance the synergy and therapeutic effect in chronic rhinosinusitis cases, a composition was tested that included rifamycin together with “Fluticasone” as a topical nasal corticosteroid. Taking into account the contribution offered by Oxymetazoline (David Brown et. al. 2011. Oxymetazoline adds to the effectiveness of fluticasone furoate in the treatment of perennial allergic rhinitis. Rhinitis, Sinusitis, and Upper Airway Disease. Volume 127, Number 4, PP. 927-934. Abril de 2011 rnacleri@surgery.bsd.uchicago.edu) and in order to achieve a comfortable, rapid decongestant effect, decongestant agent “Oxymetazoline” is incorporated into the composition. The dose of oxymetazoline used is 10 times lower than that of commercially available products containing the same when administered to adults and 20 times lower when administered to children weighing up to 15 kg. In children under 15 kg, and in the case of people with pre-existing conditions, such as prostate disease, hypertension, psychiatric treatment, and others, the optional preparation without Oxymetazoline is used to avoid the potential general undesirable effects thereof.

Finally, a pool of “bacterial antigens”—sonicated and lyophilized germs—that stimulate innate immunity elements is included in the pharmaceutical composition. The biomedical reasons that led to their inclusion are related to the biofilm and the innate immunity-stimulating activity of peptides “Defensins and Cathelicidins.” Their mechanism of action relies on the fact that positively charged peptides, Cathelicidins and Defensins, whose formation and release are stimulated by bacterial products, disrupt the negatively charged microbial membrane, hindering the replication process or causing the death of the microorganism. Their activity is non-specific to antigens, making them integral innate immunity components.

“Cathelicidins are produced by neutrophils and by the respiratory, digestive, and skin epithelia. Cathelicidins are synthesized as a precursor protein with two domains that an enzyme cleaves into two peptides, both with antimicrobial activity. Both the synthesis of the precursor and the activity of the proteolytic enzyme can be activated by certain cytokines or bacterial products. ( . . . ) Some defensins are naturally constitutively produced, but their formation is increased by stimuli from bacterial products.” (Abul Abbas, Andrew H. Lichtman, Shiv Pillai, 2022:80 in Cellular and Molecular Immunology, 10th. edition).

The results of clinical research trials with patients suffering from Chronic Rhinosinusitis were very positive, so administration and prescription thereof are a matter of routine for patients with “Chronic Rhinosinusitis.” On the other hand, Rosette C, Mazzetti A, Camerini R, Moro L, Gerloni M. Rifamycin SV Anti-inflammatory and Immunomodulatory Activities for Treatment of Mucosal and Liver Inflammation. J Infectiology. 2019; 2 (4): 18-24 Journal of Infectiology. https://doi.org/10.29245/2689-9981/2019/4.1145) and Caridad Rosette, Frances J. Agan, Niccolette Rosette, Luigi Moro, Alessandro Mazzetti, Cesare Hassan, Mara Gerloni (Rifamycin SV exhibits strong anti-inflammatory in vitro activity through pregnane X receptor stimulation and NFκB inhibition. Drug Metabolism and Pharmacokinetics. Vol. 34, Issue 3, June 2019, Pages 172-180. doi.org/10.1016/j.dmpk.2019.01.002), describe how Rifamycin inhibited cytokines by more than 90% compared to the positive control steroidal anti-inflammatory agent, dexamethasone (Dex) at 1 μM, and inhibited up to 83% of IL17A secreted by these primary immune cells. “Rifamycin showed strong downregulation of inflammatory cytokine secretion (IL2, IL6, IL8, IL17A, IFNγ and TNFα) from human colon epithelial cells as well as human immune system cells (CD4 T cells) (from peripheral blood), thus defining the in vitro anti-inflammatory characteristics of rifamycin.” “Such strong inhibition of several inflammatory cytokines in primary non-transformed human cells, in the same range as a steroid drug, further proves rifamycin as a therapeutic agent for treating mucosal inflammation in the context of IBD or IBS.”

The scientific work of these authors provides fundamental elements to understand a novel action of rifamycin, which downregulates the genes that produce inflammatory cytokines IL2, IL6, IL8, IL17A, IFNγ, and TNFα in human immune system cells (CD4 T cells). Thus, rifamycin, in addition to its local antibacterial effects, also has this immunomodulatory and anti-inflammatory action, which, in combination, explains the highly beneficial clinical effects in patients treated with this pharmaceutical composition and supports the rationale for this invention.

Therefore, it is appreciated that the presence of rifamycin would largely be responsible for the beneficial effects seen in patients using the topical nasal pharmaceutical composition of the present invention. Therefore, on the basis of the knowledge resulting from this research, the use of rifamycin in the spray composition would attenuate or control the inflammatory mechanisms that occur in the following situations, all of which are potential causes of rhinosinusitis:
1) Cellular damage or stress: produced by physical or mechanical effects such as particle inhalation ¿, initiated by alarmins such as: TSLP (thymic stromal lymphopoietin); thus, situations of cellular damage or stress and activation of DAMP receptors stimulate innate lymphoid cells (ILC2), which specifically determine TH2 profiles, and the release of IL5 and IL13. Innate lymphoid cells 1 (ILC1), which determine TH1 cytokine profiles; or cells (ILC3) with TH17 cytokines, which determine this profile.

These profiles and the activities of their cytokines trigger inflammation and its consequences. TH17 activity is characterized by the predominance of neutrophils which are present in large numbers in the cytology of nasal discharge in hyperreactive rhinosinusitis.
2) Germ antigen determinants: which are recognized by both PAMP (TLR) and DAMP receptors present mainly in antigen-presenting cells and therefore can play a role in the mechanisms of adaptive hypersensitivity, especially TH1 and TH17.
3) Innate Immunity Activation of the Inflammasome: this is a protein complex that functions as an endocellular receptor and can be activated by intracellular microbial antigens, urate microcrystals (gout), Silicon, Asbestos (lung diseases), Potassium output from the cell, also waste and substances that are produced in diseases such as Alzheimer's disease, Atherosclerosis, Metabolic Syndrome, Diabetes, Obesity are also activators of the Inflammasome, whose function is to generate IL1 and other interleukins, which make up the famous “cytokine storm”, which for example is triggered by LPS from Gram (−) bacteria (Abul K. Abbas et al., 2022, Immunology).
4) Neutrophil extracellular traps (NET) and eosinophil extracellular traps (EET): this innate immunity phenomenon occurs when the presence of germs or the components thereof cannot be eliminated by other mechanisms such as phagocytosis, neutrophils and eosinophils; under these circumstances, they expel the enzymatic components of their granules to the outside of the cell to eliminate this microbial presence in the tissues by means of these NET or EET traps that generally have a structure formed by chromatin or DNA; in this web, the granules and the content of powerful enzymes thereof are inserted which in addition to attacking the germs produce significant damage in the tissues which is expressed by inflammation.

Although there are no definitive scientific conclusions on this issue up to date, it can be considered that the presence of germs potentially activates any of the aforementioned mechanisms, since their presence, in a predisposing genotype and phenotype, can trigger inflammation dependent on innate lymphoid cells (ILCs), as well as the activation of PAMPs, DAMPs (TLRs present on antigen-presenting cells), or the inflammasome or NET or EET traps. The consequence of any of these mechanisms is the release of proinflammatory cytokines, a situation in which rifamycin shows (according to recent research) strong downregulation of the secretion of proinflammatory cytokines IL2, IL6, IL8, IL17A, IFNγ, and TNFα. This immunomodulatory and anti-inflammatory action of rifamycin would explain much of the positive and successful results observed in patients who have used and continue to use the pharmaceutical composition of the present invention; these actions of Rifamycin would, in turn, act as a potentiating effect on the anti-inflammatory actions of Fluticasone and the decongestant effects of Oxymetazoline.

### SUMMARY OF THE INVENTION

Therefore, the object of the present invention is a topical pharmaceutical composition for nasal application for the treatment and prophylaxis of Chronic Rhinosinusitis (CRS), said composition comprising a topical antibiotic agent known as Rifamycin, an anti-inflammatory agent known as Fluticasone, a decongestant agent known as Oxymetazoline, and local immunostimulants known as lyophilized bacterial antigens.

Preferably, the topical pharmaceutical composition of the present invention comprises an agent such as Rifamycin at a ratio of 130-230 mg/100 ml of the composition.

Also preferably, the topical pharmaceutical composition of the present invention further comprises a synthetic trifluorinated glucocorticosteroid such as fluticasone propionate at a ratio of 15-35 mg/100 mL of the composition.

More preferably, the topical pharmaceutical composition of the present invention optionally comprises a nasal decongestant sympathomimetic agent such as oxymetazoline hydrochloride at a ratio of 1 to 7 mg/100 ml of the composition. This component is optional, since the composition of the invention is exemplified with and without this compound, depending on whether it is used for treating children or adults.

Furthermore, the topical pharmaceutical composition of the present invention preferably comprises bacterial antigens in suspension obtained from dead bacteria subjected to ultrasonic vibration, wherein 1 mL of the bacterial antigen suspension comes from bacteria that are selected from the group of respiratory germs consisting of: Streptococcus pneumoniae, about 200 million per 100 mL of the suspension; Branhamella catarrahalis, about 100 million p/100 mL of the suspension; Haemophilus influenzae, about 200 million p/100 ml of the suspension; Streptococcus (respiratory), about 100 million p/100 ml of the suspension; Klebsiella pneumoniae, about 200 million p/100 mL of the suspension; y Micrococcus (respiratory), about 200 million p/100 mL of the suspension.

In a preferred embodiment, the topical pharmaceutical composition of the present invention comprises a topical antibiotic such as rifamycin, an anti-inflammatory agent such as fluticasone, a decongestant such as oxymetazoline, and agents such as bacterial antigens, further comprising a pharmaceutical vehicle such as 0.9% sodium chloride saline solution at a ratio of between 15 and 30 mL per 100 ml of the composition. In a preferred embodiment, the topical pharmaceutical composition of the present invention comprises a topical antibiotic such as rifamycin, an anti-inflammatory agent such as fluticasone, a decongestant such as oxymetazoline, and agents such as bacterial antigens, in a homogeneous aqueous nasal spray form.

### DETAILED DESCRIPTION OF THE INVENTION

### Definitions

“Sprayable” composition, within the context of the invention, means that the composition is administered from a spraying device or sprayer, vaporizer, or atomizer, which is inserted into the nose for nasal application. The sprayer used in this case delivers 0.10 mL of the prepared solution per spray.

The term “absorption” within the meaning of the invention refers to the movement of a therapeutic formulation into the bloodstream or a target tissue. A drug must be introduced through some route of administration, for example, nasal, oral, topical, or dermal, or in a specific dosage form, such as a tablet, capsule, or liquid. The formulations of the invention act primarily locally in the nasal cavity; their overall effects, if present, are minimal or undetectable.

The expression: “effective amount” within the context of the invention refers to an amount of therapeutic formulation that is effective at the doses and for the periods of time necessary to achieve the desired therapeutic or prophylactic result. It may vary depending on factors such as the disease state, age, gender, and weight of the individual, and its ability to elicit a desired response in the individual. A therapeutically effective amount can be measured, for example, on the basis of faster recovery, improvement, or elimination of symptoms, or other acceptable biomarkers or surrogate markers. A therapeutically effective amount is also one in which any toxic or harmful effects of the therapeutic compound are minimized and outweighed by the therapeutically beneficial effects.

In the case of the composition of the present invention, the sum of the positive effects of the active ingredients or components thereof is very evident, despite the fact that these active ingredients are present in lower doses, and some of them (for example, oxymetazoline) are much lower than those typically used in products containing them.

This results in a dual quality: the positive effects of the active ingredients thereof (anti-inflammatory, decongestant) are enhanced, and the potential harmful effects of each of them are ameliorated or diminished.

“Allergies” in the sense of the invention refer to immune system hypersensitivity disorders. Symptoms may include red eyes, itching, runny nose, eczema, hives, or asthma. Allergic reactions occur when a person's immune system reacts to normally harmless substances in the environment, food, medications, and the like. A substance that triggers an immunological reaction and generates a specific antibody against that substance is called an antigen. Allergic reactions, properly speaking, are those in which the antigen produces a reaction mediated by the antibody called “Immunoglobulin E” (IgE).

“Patient response” or “outcome” within the context of the invention refers to any endpoint indicating a result that can be evaluated clinically, radiologically, or at the laboratory.

A “pharmaceutically acceptable vehicle” within the meaning of the invention refers to a mixture of active ingredients in an aqueous phase, all of them with the addition of normal saline solution, resulting in an aqueous slightly viscous mixture with a mild, homogeneous aroma and an orange color due to the inclusion of Rifamycin, commonly included in a particular form of pharmaceutical composition. Pharmaceutically acceptable carriers are well known in the art and include, for example, aqueous solutions, such as the one of the present invention, which includes normal saline solution.

“Rhinitis” in the context of the invention refers to an inflammatory condition of the tissue lining the nasal passages, known as the mucous membranes. This inflammation can be due to several causes; some are recognized, such as upper respiratory tract infections, allergies, hay fever, and irritation from inhaled particles or pollutants. In these cases, the emergence of pathogenic microorganisms or irritants or pollutants provokes an innate immune and inflammatory reaction, depending on the magnitude and duration of the infection or contact. These clinical symptoms are generally transient, and once the infection or the cause that triggered the reaction is resolved, everything returns to normal.

Other times the clinical picture persists and without solution of continuity it becomes a Hyperreactivity phenomenon since in these cases the causes are not so well identifiable or defined; and it is under these circumstances that the pathology does not have a specific external agent as the main origin. Instead, the primary reason results from epithelial barrier integrity loss due to different causes, this triggering breakdown of peaceful coexistence between the subject, i.e. his/her immune system, and microorganisms of common or habitual presence in the nasal passages, a phenomenon known as hyperreactivity, characterized by an inappropriate reaction of the body's defense system, thus affecting such peaceful coexistence. This exaggerated reactivity by the immune system alters its normal behavior, which is “always in search of balance or homeostasis in the equation between its two variables: tolerance and reaction.” Hyperreactivity, with tolerance loss or reduction, causes the release of proinflammatory cytokines, which produce inflammation along with the symptoms and discomfort we call “Hyperreactive Rhinitis.” Depending on several parameters, rhinitis can be classified according to the duration thereof, namely, Acute and Chronic; or by its probable etiology: Infectious, Allergic, or Hyperreactive.

Generally, the most frequent symptoms are: hypersecretion, nasal congestion with obstruction, and the feeling of pressure in the rhinosinusal area. The same process can be developed in the cavities adjacent to the nasal passages, called paranasal sinuses. These are air spaces behind the cheekbones, between the eyes, and behind the forehead, all located next to the nose, hence their name “paranasal sinuses.” Normally, the paranasal sinuses drain into the nose through small openings called ostia. Any of the conditions mentioned above for rhinitis can cause inflammation, congestion, and mucus hypersecretion, blocking the flow of air and secretion, causing mucus accumulation in the paranasal sinuses, in which case we speak of sinusitis. The symptoms caused by sinusitis can be quite uncomfortable and may include: facial pain, pressure, congestion or fullness, difficulty in breathing through the nose, discharge of yellow or green mucus from the nose, pain at the tooth socket, smell and/or taste loss, headache, fatigue, sore throat, bad breath, and discomfort that, depending on the intensity, can sometimes be debilitating for performing normal tasks. It is very common for rhinitis and sinusitis to occur simultaneously, so it is appropriate to refer to them as rhinosinusitis. Currently, the term “rhinosinusitis” has replaced the term “sinusitis,” since, as mentioned herein, symptoms of both rhinitis and sinusitis coexist in most patients. “CRS is defined as persistent and/or recurrent inflammation of the nose and paranasal sinuses with the presence of two or more symptoms, one of which should be nasal congestion or runny nose, anterior/posterior nasal drip i.e. mucus running down the throat, other symptoms being facial pain/pressure, reduction or loss of smell for at least 12 weeks” [W. Fokkens, V. Lund and J. Mullol, European Position Paper on Rhinosinusitis and Nasal Polyps”, Rhinology, vol. 50, no. 1, pp. 1-12, 2012].

A “well-known spraying device” within the context of the invention comprises a storing chamber, which in the case of this study is 20 mL. The spraying device delivers 0.10 mL per spray. To activate the device, the container is manually pressed downward, with the spraying orifice facing upward and applied to the entrance of the nostril.

The present invention relates to a novel topical nasal pharmaceutical composition for the treatment and prophylaxis of Chronic Rhinosinusitis (CRS). It shows antibacterial, anti-inflammatory, decongestant, and local immunomodulatory activity, stimulating defensins and cathelicidins (antimicrobial cationic peptides). The formula contains a homogeneous nasally sprayable aqueous mixture with the following known active ingredients:
a) Rifamycin: 130 mg-230 mg/100 mL (a well-known semi-synthetic antibiotic for topical use with local antibacterial action, immunomodulatory activity, and local anti-inflammatory activity). Rifamycin is a broad-spectrum antibiotic belonging to the ansamycin family. It has potent antimicrobial and/or anti-infective activity, both locally and parenterally. Its activity has also been evaluated in vitro at minimal concentrations (μg/mL) against Gram-positive bacteria. It acts as an inhibitor of DNA-dependent RNA polymerase (DNA-RNA polymerase), blocking bacterial transcription. Its favorable pharmacokinetics and safety profile turn it into a valuable option for the treatment of serious infections (World Health Organization. Rifampicin, 2023).
b) Fluticasone Propionate: 15 mg-35 mg/100 mL (a well-known anti-inflammatory drug for local use).
c) Oxymetazoline hydrochloride: 1 mg/100 mL to 7 mg/100 mL (a well-known imidazole derivative with sympathomimetic properties and strong vasoconstrictor action that produces a detumescent effect at the delivery site). Its use is optional for the treatment of children weighing less than 15 kg or patients with pre-existing conditions.
d) Bacterial antigens: 0.25 to 0.35 mL of a bacterial antigen suspension consisting of a suspension of dead bacteria subjected to ultrasonic vibration, wherein said bacteria are selected from respiratory germs. In particular, 1 mL of said suspension provides: Streptococcus pneumoniae, about 200 million per 100 mL of the suspension; Branhamella catarrahalis, about 100 million p/100 ml of the suspension; Haemophilus influenzae, about 200 million p/100 ml of the suspension; Streptococcus (respiratory), about 100 million p/100 ml of the suspension; Klebsiella pneumoniae, about 200 million p/100 ml of the suspension; y Micrococcus (respiratory), about 200 million p/100 ml of the suspension.
e) The preferred dosage range indicated for each of the active ingredients guarantees the greatest therapeutic efficacy of the composition, offering an advantage over the administration of the active ingredients alone. The inclusion of these well-known active ingredients also guarantees the stability, efficacy, and spraying conditions of the composition. To ensure the bioavailability and stability of the composition, a clear and colorless 0.9% sodium chloride saline solution, free of visible particles, sterile, and non-pyrogenic, is used at a ratio of 15 to 30 mL per 100 mL of the composition. This prevents the growth of bacterial or fungal cultures, allowing for improved bioavailability of the active ingredients. It is estimated that the composition shelf-life under stable temperature and other physical-chemical conditions is 12 months. It is noted that the expiration dates for each of the products comprised by the solution must also be later than this 12-month period.

Regarding the therapeutic action thereof, the composition has proven to be highly effective for the treatment and prophylaxis of rhinitis and rhinosinusitis. With the aim of minimizing potential adverse effects and reactions during the therapeutic use of the composition in vulnerable patient populations, those at greater risk, or those with pre-existing diseases and/or conditions, a usage protocol was developed based on the age, weight, or pre-existing pathologies of each patient:
a) In adult patients without pre-existing conditions or children weighing more than 30 kg, oxymetazoline hydrochloride is added to the composition at a rate of 3-7 mg/100 mL, approximately 10 times lower than commercial spray products containing it. The use thereof is avoided in patients with prostate, cardiac, hypertensive, or neuropsychological disorders.
b) In children weighing more than 15 kg and less than 30 kg, oxymetazoline hydrochloride is added to the composition at a lower rate, between 1.5 and 3 mg/100 mL, approximately 10 times lower than commercial spray products for children containing the same.
c) The composition does not directly include Oxymetazoline Hydrochloride in the formula for children weighing less than 15 kg or for adults with pre-existing conditions. It is also highlighted that the use of the composition simultaneously with other inhalant medications is inconvenient.

If the composition is not used according to the instructions, the risks may vary. Despite this, there have been no cases of overdose reported, nor have there been any problems with eventual dose duplication. In the event of overdose, the patient should immediately go to the nearest hospital or contact a Toxicology Center.

Regarding the best method of administration for the composition, nasal spraying is preferred due to its effectiveness at the specific site of action, taking into account certain criteria that have been standardized and systematized over time. For the product containment, it was decided to use a commercial neutral container with a maximum capacity of 20 mL, filled with 16.5 ml of a homogeneous solution containing the active ingredients. The quality of the container and the contents thereof were always supervised by a licensed pharmaceutical professional from Río Cuarto City, Argentina. This professional was responsible for controlling and ensuring the entire composition manufacturing process, the quality, and the hermetic sealing of the product from the very beginning. Each container has a device for spraying small droplets with a nasal applicator, ranging from 0.5 to 0.15 mL per spray or dose. The container operates using a manual dosing pump. Along with the container and the instructions thereof, a small glossary with the basic instructions for use and administration of the nasal applicator was designed and incorporated into the packaging box. The necessary requirements to achieve the desired effect are detailed as follows: i) clean the nasal passages, removing all secretion; ii) shake the device well before use; iii) remove the applicator cap; iv) the applicator is now ready for use; v) place the applicator with its orifice just at the entrance of the nostril, taking care that it does not touch any tissue, for when it comes into contact with tissue, a drop will be released instead of the spray in droplets, which is the desired effect; vi) apply pressure to the device to release a 0.10 mL dose of the composition contents; if the patient feels that the spraying has been correct, proceed with the same action in the other nostril; if a feeling of severe obstruction occurs, inhalation may be performed at the moment of spraying; vii) lie down for a moment, 30 seconds to a minute, to better distribute the medication in the nasal passages and sinuses; and viii) after use, clean the applicator with a tissue and replace the cap; close the container with the cap and store it in a place protected from the sun at room temperature; it is also recommended not to share or exchange the container with another patient.

### EXEMPLARY EMBODIMENT

An exemplary embodiment of the composition is the one described below, “nasal solution composition for adults” comprising a combination of:
180 mg Rifamycin per 100 mL of the composition (or 0.180 g/100 mL);
27 mg Fluticasone Propionate per 100 ml of the composition (or 0.027 g/100 mL);
4.5 mg Oxymetazoline per 100 ml of the composition (or 0.0045 g/100 mL);
0.3 mL bacterial antigen suspension per 100 mL of the composition, which antigens are obtained from dead bacteria subjected to ultrasonic vibration,
18.70 mL 0.9% saline solution per 100 mL of the composition, and
pharmaceutically acceptable excipients q.s.;
wherein 0.30 mL of bacterial antigen suspension/100 ml of the composition provides antigens from respiratory germs selected from: Streptococcus pneumoniae 60 million per 100 ml of the composition; Branhamella catarrahalis 30 million per 100 mL of the composition; Haemophilus influenzae 60 million per 100 mL of the composition; Streptococcus (respiratory) 30 million per 100 mL of the composition; Klebsiella pneumoniae 60 million per 100 mL of the composition; and Micrococcus (respiratory) 60 million per 100 mL of the composition.

Pharmaceutically acceptable excipients in nasal sprays provide stability, safety, efficacy, and patient comfort. Pharmaceutically acceptable excipients for nasal use must be compatible with the nasal mucosa, non-irritating and non-toxic, and they should ensure adequate drug release and absorption at the treatment site.

Pharmaceutically acceptable excipients can be identified as vehicles that dissolve or suspend the active ingredient, such as purified water, isotonic saline solution, i.e. 0.9% sodium chloride, and mixtures thereof; viscosity and bioadhesive agents to increase the contact time of the drug with the nasal mucosa, improving its absorption and prolonging its effect, selected from: microcrystalline cellulose and sodium carboxymethylcellulose, polymers such as Carbopol® or hydroxypropyl methylcellulose (HPMC), hyaluronic acid, and mixtures thereof; buffering agents (buffers) to maintain the pH within a suitable range, generally between 5.5 and 7.5, for drug stability and to prevent mucosal irritation selected from monosodium and disodium phosphate, citric acid or hydrochloric acid with sodium hydroxide for pH adjustment; preservatives to prevent microbial growth and maintain sterility of the product during use, selected from: benzalkonium chloride, parabens such as methylparaben and propylparaben, phenylethyl alcohol, disodium edetate (EDTA), and mixtures thereof; wetting and solubilizing agents to improve the solubility of the active ingredients and ensure homogeneous dispersion while preventing dryness of the nasal mucosa selected from: polysorbates (such as Polysorbate 80 or Tween 80), propylene glycol and polyethylene glycols (PEG), glycerin, and mixtures thereof; bulking agent or sweetener selected from anhydrous glucose, dextrose, sucralose, and mixtures thereof; and isotonizing agents that prevent irritation and damage to nasal mucosa cells, such as sodium chloride, which may be provided by the isotonic saline solution vehicle.

Pharmaceutically acceptable excipients are added to the formulation in amounts sufficient to achieve the desired effects and benefits, and are within the reach of a person skilled in the art.

The density of the pharmaceutical composition of the present invention is between 1.015 g/cm³ and 1.025 g/cm³, preferably 1.020 g/cm³.

### Exemplary Applications

The preferred method of administration uses a nasal sprayer that sprays the composition once or twice daily at a dosage ranging from 0.10 mL to 0.20 mL per dose. It is suggested that the composition be administered preferably in the morning and evening at the beginning of treatment for 10 to 30 days depending on the clinical picture. It is then continued at night for as long as required by the patient's condition. No adverse effects have been observed in patients using it continuously for long periods of time, up to more than one year. To benefit from the effective action according to the preferred use of the invention, the prescribed instructions before, during, and after use must be taken into account. These include product and patient asepsis. To ensure the microbiological stability of the composition even after opening, the product is stored in a protected place at room temperature (no higher than 25° C.).

The composition has demonstrated therapeutic efficacy when administered in important and complicated clinical pictures of CRS in investigated patients, cases of pathologies of years of evolution, sometimes with polyps, or pictures of persistent purulent secretions of prolonged evolution, rebellious headaches, habitual cough for months in children and adults, secondary skin reactions due to rhinosinusitis, chronic conjunctivitis caused by rhinosinusitis, many of them having previously used other medications failing to obtain positive responses (Fluticasone, Mometasone, Budesonide, all in nasal spray), associations (Fluticasone-Oxymetazoline, Fluticasone-Azelastine, Neomycin-Dexamethasone Naphazoline-Diphenhydramine all in nasal spray); and other oral medications Montelukast. Antihistamines—Betamethasone and antibiotics—were among the most frequently used in the patients examined; however, the present pharmaceutical composition solved or provided very favorable improvements in the vast majority of cases. Avoiding polyp surgeries, allowing parents of children affected by persistent cough due to rhinosinusitis to sleep soundly, eliminating coughs in coughers due to CRS, restoring nasal breathing in children who mouth breathe, making coughs disappear in children who play sports, eliminating the use of nasal decongestants in adults when breathing through the nose again, restoring the sense of smell to those who had lost or diminished it due to CRS, eliminating headaches or discomfort in the rhinosinusal region, positive modification in the frequency and intensity of asthma attacks, these are just to name a few out of the many favorable results observed.

With these very positive results in complex clinical conditions, in which the other aforementioned medications were ineffective, the composition of the present invention yielded positive and often surprising responses. These evident clinical realities with the patients studied are the main motivation for this filing, based on the conviction that the Formula of the pharmaceutical composition of the present invention is a superior contribution to current topical nasal medication and will make an important contribution to improving the efficacy of therapeutic tools for patients suffering from Chronic Rhinosinusitis.

### Statistical Clinical Review of Chronic Rhinosinusitis (CRS)

### Evaluation of Medical Records and Outcomes Using SNOT22 for the Use of the Nasal Spray Pharmaceutical Composition in Adult and Pediatric Patients.

1) Premises and concepts considered in the creation of the Formula of the pharmaceutical composition.
2) Protocol Form for Selection of Medical Records.
3) Baseline Conditions and Materials.
4) TABLE of outcomes in adults.
5) Results of the use of other products prior to the use of the pharmaceutical composition of the invention in adults.
6) TABLE of outcomes in children and adolescents up to 17 years of age.
7) Results of the use of other products prior to the use of the pharmaceutical composition of the invention in children and adolescents up to and including 17 years of age.
8) Conclusions.

### 1) PREMISES AND CONCEPTS CONSIDERED IN THE CREATION OF THE FORMULA OF THE PHARMACEUTICAL COMPOSITION

Before using the pharmaceutical composition in patients, certain medical-clinical-ethical criteria to be taken into account were established for the formula of the composition. In brief, the premises are the following:
a) Balance or homeostasis in the ongoing relationship between the patient's immune system and the microbial flora of the rhinosinusal microenvironment should be attempted.
b) Active ingredients with extensive international experience should be employed.
c) The pharmaceutical composition must pose no risk to patients.
d) The pharmaceutical composition to be employed must be easy to use.
e) The preparation thereof must be accessible within the scope of pharmacotechnical practice.

### 2) PROTOCOL FORM FOR SELECTION OF MEDICAL RECORDS

A protocol was then drawn up with essential requirements and situations common to all medical records.

This protocol form especially specified the clinical data collected during the initial consultation, including: reason for consultation, symptoms and signs, characteristics of the symptoms, duration of symptoms, previous studies and treatments, medications used, and other data that may be related to rhinosinusitis, such as occupation, transportation, family history, place of residence, and any additional information.

The filled-in form contains the clinical diagnosis of radiologically confirmed CRS and the assessment of laboratory studies and other relevant data. Periodic monitoring of the evolution over time is carried out during the various consultations.

To this form, it was added the International SNOT22 Measurement System (SNOT, Sino-Nasal Outcome Tests). The SNOT22 system refers to 22 possible signs or symptoms that may occur in patients with Chronic Rhinosinusitis (CRS) (see: Valdés P., Constanza, Muñoz M., Tamara, Barría E., Tamara, Bogado B., Mariana, Ruz M., Paula, Cardemil M., Felipe, Ruz G., Samanta, & Veloz T., Marcela. (2020). Validación de encuesta de calidad de vida “SNOT-22” en población chilena. Revista de otorrinolaringología y cirugía de cabeza y cuello, 80(4), 425-433. https://dx.doi.org/10.4067/S0718-48162020000400425; Hopkins, C., Gillett, S., Slack, R., Lund, V. J., & Browne, J. P. (2009). Psychometric validity of the 22-item Sinonasal Outcome Test. Clinical otolaryngology, 34 (5), 447-454. https://doi.org/10.1111/j.1749-4486.2009.01995.x).

Thus, when describing the patient's clinical picture and symptoms, the most prominent signs or symptoms are recorded in the form, assessing their persistence, frequency, and intensity. They are scored with a number from 0 to 5 depending on the importance of their clinical characteristics. A 5-point score is assigned if their importance is maximum, either in terms of persistence, frequency, and intensity. A 0-point score is given if the symptom has disappeared, or the corresponding points are given based on the assessment performed at that time of this same symptom after treatment with the nasal pharmaceutical composition of the invention. Each sign or symptom is then assessed on a scale ranging from 0 (asymptomatic) to 5 (very severe), depending on the severity or relevance of the discomfort or condition.

At the final follow-up, the total number of symptoms assessed may yield the following outcomes: a) 0 to 1, Excellent, b) 1 to 2, Very Good, c) 2 to 3, Good, d) 3 to 4, Poor and e) 4 to 5, No beneficial results.

Once the form was completed and with the aim of carrying out a research project on outcome assessment, 450 medical records were selected from a plurality of cases of patients treated, resulting in the selection for the research of all those cases whose SNOT22 score at onset was equal to or greater than a score of 3.50 points, that is, clinical pictures of rhinosinusitis in which the baseline condition was poor-bad or bad.

On the one hand, 220 patients of both genders aged 18 and over and adults with CRS were selected and investigated. All of them were treated with the pharmaceutical composition of the invention.

On the other hand, 230 medical cases of children and adolescents of both genders up to and including 17 years of age were treated with the pharmaceutical composition in nasal spray.

### 3) BASELINE CONDITIONS AND MATERIALS

For the measurement and assessment of the outcomes exhibited in Table 1 and Table 2 below, it was essential that all adult patients had been clinically and radiologically diagnosed with Chronic Rhinosinusitis or, in the case of children, Chronic or Persistent Rhinosinusitis, always with evolution for more than 3 months.

Essentially, it was considered that all investigated and assessed patients had a SNOT22 Baseline Evaluation Score equal to or greater than 3.50; that is, a diagnosis of poor-to-bad to bad onset, complicated, and complex cases. In this way, the score achieved at the final follow-up is compared with the baseline score.

Thus, on the basis of the comparison of the SNOT22 Baseline and the SNOT22 Final Evolution Control, numerical data arise that indicate the clinical outcome of the treatment carried out, measured by the numerical difference between the baseline and final control.

### Formulations of the Pharmaceutical Compositions Used

### i) Formula of the Pharmaceutical Composition in Nasal Solution for Adults:

i) The pharmaceutical composition in nasal solution for adults comprises:
Rifamycin: 180 mg/100 mL of the composition;
Fluticasone propionate: 27 mg/100 mL of the composition;
Oxymetazoline hydrochloride: 4.5 mg/100 mL of the composition;
Bacterial antigen solution: 0.30 mL/100 mL of the composition;
0.9% saline solution; 18.70 mL/100 mL of the composition; and
Pharmaceutically acceptable excipients q.s.;
Density about 1.020 g/cm³, pH about 7.48.

### ii) Formula of the Pharmaceutical Composition in Nasal Solution for Children (Children Between 15 kg and 30 kg in Weight):

The pharmaceutical composition in nasal solution for children comprises:
Rifamycin: 180 mg/100 mL of the composition;
Fluticasone propionate: 27 mg/100 mL of the composition;
Oxymetazoline hydrochloride: 2.25 mg/100 mL of the composition;
Bacterial antigen suspension: 0.30 mL/100 mL of the composition;
0.9% saline solution; 23.20 mL/100 mL of the composition; and
Pharmaceutically acceptable excipients q.s.;
Density about 1.020 g/cm³, pH about 7.08.
   iii) Formula of the Pharmaceutical Composition in Nasal Spray without Oxymetazoline (for Children Weighing Less than 15 kg and Adults with Pre-Existing Conditions):

The pharmaceutical composition in nasal solution for toddlers and adults with pre-existing conditions comprises:
Rifamycin: 180 mg/100 mL of the composition;
Fluticasone propionate: 27 mg/100 mL of the composition;
Bacterial antigen solution: 0.3 mL/100 mL of the composition;
0.9% saline solution; 27.70 mL/100 mL of the composition; and
Pharmaceutically acceptable excipients q.s.;
Density about 1.020 g/cm³, pH about 6.10.

### 4) TABLE OF OUTCOMES IN ADULTS

TABLE 1
Assessment of therapeutic response after use of the pharmaceutical composition in young people of 18 years
of age and older and adults, both genders, among 220 patients evaluated. Sample evaluated at the last
consultation or treatment follow-up after using the pharmaceutical composition of the invention.

| | Therapeutic | | |
|---|---|---|---|
| | response | Characterization | Universe |
| A | EXCELLENT | In many cases, the young and/or adult patient experiences complete | 30% |
| | 66 Patients | disappearance of symptoms within 1-7 days of composition | |
| | SNOT22 | administration after 3 or more months of persistence of the symptoms | |
| | 0.00 to 1.00 | that led to medical consultation. Other chronic cases, years evolving, | |
| | | used only at night. Nasal asymptomatic for several months. | |
| B | VERY GOOD | The patient reports disappearance or a very significant decrease in the | 38% |
| | 82 Patients | frequency and intensity of the clinical symptoms. This is applicable to | |
| | SNOT22 | patients who have tried other therapeutic alternatives without positive | |
| | 1.00 to 2.00 | results, or cases of intense and prolonged discomfort that required | |
| | | ongoing and/or repeated treatments with little or no change. The patient | |
| | | continues using a single dose at night as maintenance therapy. | |
| C | GOOD | a) The patient experiences a favorable change in the frequency and | 19% |
| | 42 Patients | intensity of symptoms from the initial clinical picture; symptoms | |
| | SNOT22 | reappear upon discontinuation of the compound. | |
| | 2.00 to 3.00 | b) The use of other decongestants and/or oral or topical steroids | |
| | | administered at high doses and frequency is replaced by a single | |
| | | nighttime dose of the compound. | |
| D | POOR | The patient shows some change in the intensity and frequency of the | 8% |
| | 19 Patients | clinical picture, upon discontinuation the initial discomfort persists: | |
| | SNOT22 | cough, nasal congestion, nasal discharge. | |
| | 3.00 to 4.00 | | |
| E | NO BENEFITIAL | a) The patient shows no substantial changes in their clinical picture. | 5% |
| | RESULT | b) In other cases: nasal irritation, nasal burning, and sneezing when | |
| | 11 Patients | applying the product prevent its use. | |
| | SNOT22 | | |
| | 4.00 to 5.00 | | |

| | |
|---|---|
| Total | 100% |

As shown in Table 1, the outcomes are very satisfactory. Evidence of improvement occurs from the moment the treatment began. The composition is well-tolerated, it generally acts without irritation, and no cases of rebound congestion have been reported after administration for more than 30 days. Scarse mild and temporary adverse effects (between 2 to 3% of the cases) observed include nasal discomfort such as burning, stinging, irritation, sneezing upon application of the nasal spray, and very rarely, epistaxis. At the indicated doses and as prescribed, no cases of contact dermatitis or Candida nasopharyngitis have been reported associated with the intranasal use of Fluticasone and Rifamycin, nor any reaction that could be assumed to be local or general allergy in the treated patients.

SNOT 22 Application Results. Final Control Score for 220 Patients Over 18 Years of Age Assessed

### Assessment Method:

Out of the 220 cases investigated the baseline score was:

From 3.50 to 4.00 in 113 cases.

From 4.00 to 4.50 in 76 cases.

From 4.50 to 5.00 in 31 cases.

Excellent: (30%) 66 Patients. Outcome: SNOT22 from 0 to 0.99 points, final control.

This group showed rapid disappearance of persistent, daily symptoms within a few days of using the Nasal Solution. Improvement persisted 90 days after the end of treatment. Others, using the pharmaceutical composition of the present invention only once a day, remained asymptomatic for several months.

Very good: (40%) 82 Patients. Outcome: SNOT22 from 1 to 1.99 points, final control.

In these patients, their complicated condition is specifically assessed at the initial consultation and compared with the final follow-up condition. Even if they experience sporadic, non-significant discomfort, the disappearance and/or reduction of some very bothersome and persistent symptoms they suffered from, such as persistent nighttime cough, severe nasal obstruction, and the reduction in the intensity of discomfort when they began using the pharmaceutical composition of the invention, are also taken into account. While using the topical pharmaceutical composition of the invention, they did not require other medications, which, anyway, had not completely resolved their discomfort.

Good: (18%) 42 Patients. Outcome: SNOT22 from 2 to 2.99 points, final control.

This group is characterized by persistent symptoms, but with less intensity and/or less frequency, which do not affect their quality of life, but they must continue taking night-time medication.

Poor: (6%) 19 Patients. Outcome: SNOT22 from 3 to 3.99 points, final control.

Generally, patients with frequent respiratory infections, especially during cold weather. Unsatisfactory progress, with some favorable changes but with persistence of other significant discomfort, for the purposes of scoring. Progress with some favorable changes of little significance.

A significant number of these cases (17) belong to the group whose Baseline Score was between 4.00 and 5.00.

Bad: (5%) 11 Patients. Outcome: SNOT22 from 4 to 5.00 points, final control.

The reasons for this outcome included: the patients could not tolerate the spray because it made them sneeze; others experienced a burning nose; and some patients showed no positive change. In some cases, the situation did not allow the underlying cause to be ruled out, such as commuting by motorcycle in winter, working in stores under cold temperature conditions, and others.

Nearly 90% of these cases (9 patients) belong to the group whose Baseline Score was between 4.50 and 5.00.

### 5) RESULTS OF THE USE OF OTHER PRODUCTS PRIOR TO THE USE OF THE PHARMACEUTICAL COMPOSITION OF THE INVENTION IN ADULTS

The following is a comparison of the outcomes obtained in adult patients treated with the pharmaceutical composition with those previously treated with other products.

Out of a total of 220 patients treated and examined, 148 patients (68%) showed excellent and very good results according to Table 1, namely:
I) Out of the 148 patients, 47 patients previously used a nasal spray of Fluticasone, Mometasone, or Budesonide with unsuccessful results.
II) Out of the 148 patients, 21 had previously used Montelukast with unsuccessful results.
III) Out of the 148 patients with excellent to very good results, 67 had previously used decongestant drops or sprays with unsuccessful results.

### 6) TABLE OF OUTCOMES IN CHILDREN AND ADOLESCENTS UP TO 17 YEARS OF AGE

TABLE 2
Assessment of therapeutic response after use of the pharmaceutical composition in children and adolescents
up to and including 17 years of age, both genders, among 230 patients evaluated. Sample evaluated at the
last consultation or treatment follow-up after using the pharmaceutical composition of the invention.

| | Therapeutic | | |
|---|---|---|---|
| | response | Characterization | Universe |
| A | EXCELLENT | The child and/or adolescent experiences complete disappearance of | 31% |
| | 71 Patients | symptoms within 1-7 days of composition administration, in many cases | |
| | SNOT22 | after 3 or more months of persistence of the symptoms that led to medical | |
| | 0.00 to 1.00 | consultation. X-rays frequently show a marked decrease in aeration. With | |
| | | treatment lasting 4 to 6 months, the radiological changes are very | |
| | | favorable. | |
| B | VERY GOOD | Within a few days, the child showed a marked decrease in the intensity and | 40% |
| | 92 Patients | frequency of very bothersome and persistent symptoms. This treatment is | |
| | SNOT22 | applicable to patients who had been alternating other medications, with | |
| | 1.00 to 2.00 | intense and prolonged discomfort, and who required ongoing and/or | |
| | | repeated treatment with other oral and/or topical medications with little | |
| | | or no improvement. Sinusitis X-rays are frequently completely veiled. These | |
| | | patients require a continuous daily dose for at least 6 to 9 months, after | |
| | | which a significant change in X-rays is required. | |
| C | GOOD | a) The child shows a favorable change in the frequency and intensity of the | 18% |
| | 41 Patients | symptoms from the initial medical picture, but these symptoms reappear | |
| | SNOT22 | upon discontinuation of the compound. b) The child shows a favorable | |
| | 2.00 to 3.00 | change in the symptoms and the possibility of replacing or reducing other | |
| | | frequently used decongestants with undesirable effects (oral decongestants | |
| | | with ephedrine, repeated nasal drops, oral corticosteroids) with a single | |
| | | nighttime dose of the compound. | |
| D | POOR | The child shows a slight improvement in his clinical condition, although | 6% |
| | 14 Patients | not enough to suspend or discontinue treatment with other oral | |
| | SNOT22 | medications. | |
| | 3.00 to 4.00 | | |
| E | NO BENEFITIAL | a) The child shows no substantial changes in his/her clinical picture. b) | 5% |
| | RESULT | Nasal discomfort, sneezing, when applying the product, which is why the | |
| | 12 Patients | child refuses to use it. c) Misuse of the spray. | |
| | SNOT22 | | |
| | 4.00 to 5.00 | | |

### Results of Applying SNOT22. Control Score in 230 Children and Adolescents Up to 17 Years of Age Evaluated.

### Assessment Method:

Out of the 230 cases investigated the starting score was:

From 3.50 to 4.00 in 129 cases.

From 4.00 to 4.50 in 79 cases.

From 4.50 to 5.00 in 22 cases.

Excellent: (31%) 71 Patients. Outcome: SNOT22 from 0 to 0.99 points, final control.

This group showed rapid disappearance of continuous, daily symptoms within a few days of using the nasal spray formula of the pharmaceutical composition. Generally, improvement persisted 90 days after the end of treatment.

Other complicated cases that did not respond to other medications, when using the pharmaceutical composition in nasal spray only once a day, were asymptomatic for several months.

Very good: (40%) 92 Patients. Result: SNOT22 from 1 to 1.99 points, final control.

In children, their complicated condition is specifically assessed at the initial consultation and compared with the follow-up condition. Even if they experience sporadic, non-significant discomfort, the reduction of very bothersome and persistent symptoms they suffered from, such as persistent nighttime cough, continuous mucus, severe nasal obstruction, and the reduction in the intensity of discomfort when they began using the pharmaceutical composition, are also taken into account. The radiological change is also evident a few months after starting treatment. Other children and young adults did not need other medications while using the pharmaceutical composition, which, anyway, had not shown to completely resolve their discomfort.

Good: (18%) 41 Patients. Outcome: SNOT22 from 2 to 2.99 points, final control.

This group is characterized by persistent symptoms, but with less intensity and/or less frequency, which do not affect their quality of life, but they must continue taking nighttime medication.

Poor: (6%) 14 Patients. Result: SNOT22 from 3 to 3.99 points, final control.

Generally, children with frequent respiratory infections, especially during cold weather. Unsatisfactory progress, with some favorable changes but with persistence of other significant discomfort, for the purposes of scoring. Progress with some favorable changes of scarse significance.

This group included mostly those who had a baseline score between 4.00 and 4.50.

Bad: (5%) 12 Patients. Outcome: SNOT22 from 4.00 to 5.00 points, final control.

The reasons for this outcome included: the patients could not tolerate the spray; it made them sneeze; others experienced a burning nose; and some patients showed scarse or no favorable change. In some cases, the situation did not allow the underlying cause of the condition to be ruled out, such as commuting by motorcycle to school in winter, and others. This group included mostly those who had a baseline score between 4.50 and 5.00.

### 7) RESULTS OF THE USE OF OTHER PRODUCTS PRIOR TO THE USE OF THE PHARMACEUTICAL COMPOSITION OF THE INVENTION IN CHILDREN AND ADOLESCENTS UP TO AND INCLUDING 17 YEARS OF AGE

Out of a total of 230 patients treated with the pharmaceutical composition and examined, 163 patients (more than 70%) showed excellent and very good outcomes according to Table 2, namely:
I) Out of the 163 patients, 43 patients had previously been treated with a nasal spray comprising Fluticasone, or Mometasone, or Budesonide with non-positive results.
II) Out of the 163 patients, 67 patients had been previously treated with Montelukast showing non-positive outcomes.
III) Out of the 163 patients, 29 had previously used decongestant drops or sprays with unsuccessful results.

### Note:

The statistics from this research group show that these patients, who did not exhibit significant outcomes with the medications commonly used in these cases, instead exhibited very good to excellent outcomes in over 70% of cases using the pharmaceutical composition of the invention in the form of a nasal spray.

In this regard, the conclusive radiological data in children is noteworthy, as the positive change between the initial X-ray and that of a subsequent follow-up is clearly evident in a significant number of cases.

The selected patients, as seen in their medical records, all with significant clinical and radiological symptoms of CRS, were medicated with the pharmaceutical composition formula in nasal spray from the beginning, applying one spray in each nostril twice a day between 15 to 30 days at the beginning, depending on their clinical picture, and in some specific cases for a longer time, then continued only at night between 1 month and 5 months according to radiological and clinical control.

In most cases, the formula of the pharmaceutical composition was administered as the sole medication; in other situations, it was accompanied by medication for pathologies also present at that time, such as an antibiotic if the patient also suffered from bronchitis, for example; and in some other patients, antigen therapy treatment was prescribed at the same time.

For periodic follow-ups, patients were seen every 30 to 60 days at the beginning of treatment, and then, depending on their progress, the follow-up periods were extended. In most cases, treatment consisted of applying the pharmaceutical composition for a period of 2 to 5 months, with very favorable outcomes observed, as shown in the statistics in Table 2.

### 8) CONCLUSIONS

The precise and appropriate combination of the active ingredients of the pharmaceutical composition of the invention produced a collaborative synergy between them that significantly enhanced the beneficial effect of said composition, surpassing the possible sum of the benefits of each of those active ingredients used individually.

Furthermore, each of these components were used in the pharmaceutical composition of the present invention in lower doses-some of them much lower-than those used in the various commercial products that contain them and are recommended for Rhinitis and Rhinosinusitis.

The numbers resulting from the outcomes of the 450 cases selected for this statistical work, treated with the pharmaceutical composition of the present invention, confirm the positive clinical evolution of the patients investigated in the order of 70% between Excellent and Very good, clearly surpassing the results of treatments with the leading standard medications for Chronic Rhinosinusitis. Many of the patients examined had been treated with these products at some point, with insufficient therapeutic results.

Analyzing the sample results, they are highly positive, novel, and unexpected compared to the prior art. Although the therapeutic effects are produced in almost all clinical forms of rhinosinusitis, including the acute form, early efficacy was noted in those related to bacterial microbes with the presence of habitual mucopurulent secretion, also in allergic profiles, and in other environmental situations that could cause innate hyperreactivity, and/or adaptive sensitivity, with Th2-Th17 profiles. There is no doubt that antibiotic Rifamycin has an important effect, in addition to the antibiotic effect, in neutralizing some pro-inflammatory processes. The secret to its therapeutic success lies in the “composition” itself, that is, in the exclusive combination of these active ingredients. Based on the results achieved, this appears to be more a multiplication than a sum of the positive effects or qualities provided by each of these active ingredients, while also demonstrating the absence of any negative effects at the doses used.

1. A topical nasal pharmaceutical composition for the treatment and prophylaxis of Chronic Rhinosinusitis (CRS) comprising: Rifamycin as a topical antibiotic agent, Fluticasone as a synthetic trifluorinated glucocorticosteroid anti-inflammatory agent, optionally Oxymetazoline as a nasal decongestant sympathomimetic agent, and a lyophilized local immunostimulatory bacterial antigen.

2. The topical nasal pharmaceutical composition according to claim 1, wherein Rifamycin is at a ratio of 130-230 mg/100 mL of the composition.

3. The topical nasal pharmaceutical composition according to claim 1, wherein Fluticasone is present as Fluticasone Propionate at a ratio of 15-35 mg/100 mL of the composition.

4. The topical nasal pharmaceutical composition according to claim 1, wherein Oxymetazoline is present as Oxymetazoline Hydrochloride at a ratio of 1-7 mg/100 mL of the composition.

5. The topical nasal pharmaceutical composition according to claim 1, wherein the lyophilized bacterial antigens are in suspension at a ratio of 0.25 to 0.35 mL/100 mL of the composition, and wherein 1 mL of the suspension provides antigens of respiratory germs selected from: Streptococcus pneumoniae 200 million per 100 ml of the suspension; Branhamella catarrahalis 100 million per 100 mL of the suspension; Haemophilus influenzae 200 million per 100 mL of the suspension; Streptococcus (respiratory) 100 million per 100 ml of the suspension; Klebsiella pneumoniae 200 million per 100 mL of the suspension; and Micrococcus (respiratory) 200 million per 100 mL of the suspension.

6. The topical nasal pharmaceutical composition according to claim 1, the composition further comprising a 0.9% sodium chloride saline solution as a pharmaceutical vehicle at a ratio of 15 to 30 mL/100 mL of the composition.

7. The topical nasal pharmaceutical composition according to claim 1, wherein the composition is in a homogeneous aqueous nasal vaporizable form.

8. A pharmaceutical composition in topical nasal solution for adults, comprising:
180 mg Rifamycin per 100 mL of the composition;
27 mg Fluticasone Propionate per 100 mL of the composition;
4.5 mg Oxymetazoline Hydrochloride in per 100 mL of the composition;
0.30 mL bacterial antigen suspension per 100 mL of the composition;
18.70 mL 0.9% saline solution per 100 mL of the composition, and
pharmaceutically acceptable excipients q.s.;
wherein 0.30 mL bacterial antigen suspension per 100 ml of the composition provides antigens from respiratory germs selected from: Streptococcus pneumoniae 60 million per 100 mL of the composition; Branhamella catarrahalis 30 million per 100 mL of the composition; Haemophilus influenzae 60 million per 100 mL of the composition; Streptococcus (respiratory) 30 million per 100 mL of the composition; Klebsiella pneumoniae 60 million per 100 mL of the composition; and Micrococcus (respiratory) 60 million per 100 mL of the composition.

9. A pharmaceutical composition in topical nasal solution for children weighing between 15 kg and 30 kg, comprising:
180 mg Rifamycin per 100 mL of the composition;
27 mg Fluticasone Propionate per 100 mL of the composition;
2.25 mg Oxymetazoline Hydrochloride in per 100 mL of the composition;
0.30 mL bacterial antigen suspension per 100 mL of the composition;
23.20 mL 0.9% saline solution per 100 mL of the composition, and
pharmaceutically acceptable excipients q.s.;
wherein 0.30 mL bacterial antigen suspension per 100 mL of the composition provides antigens from respiratory germs selected from: Streptococcus pneumoniae 60 million per 100 mL of the composition; Branhamella catarrahalis 30 million per 100 mL of the composition; Haemophilus influenzae 60 million per 100 mL of the composition; Streptococcus (respiratory) 30 million per 100 mL of the composition; Klebsiella pneumoniae 60 million per 100 mL of the composition; and Micrococcus (respiratory) 60 million per 100 mL of the composition.

10. A pharmaceutical composition in nasal spray for children weighing less than 15 kg and adults with pre-existing conditions, comprising:
180 mg Rifamycin per 100 mL of the composition;
27 mg Fluticasone Propionate per 100 mL of the composition;
0.30 mL bacterial antigen suspension per 100 mL of the composition;
27.70 mL 0.9% saline solution per 100 mL of the composition, and
pharmaceutically acceptable excipients q.s.;
wherein 0.30 mL bacterial antigen suspension per 100 mL of the composition provides antigens from respiratory germs selected from: Streptococcus pneumoniae 60 million per 100 mL of the composition; Branhamella catarrahalis 30 million per 100 mL of the composition; Haemophilus influenzae 60 million per 100 mL of the composition; Streptococcus (respiratory) 30 million per 100 mL of the composition; Klebsiella pneumoniae 60 million per 100 mL of the composition; and Micrococcus (respiratory) 60 million per 100 mL of the composition.
